(19) United States
(12) Patent Application Publication
(10) Pub. No.: US 2018/0071299 A1
(43) Pub. Date: Mar. 15, 2018

(54) METHODS FOR TREATING PSYCHOSIS ASSOCIATED WITH PARKINSON'S DISEASE

(71) Applicant: Reviva Pharmaceuticals, Inc., Santa Clara, CA (US)

(72) Inventors: Laxminarayan BHAT, Santa Clara, CA (US); Marc CANTILLON, Santa Clara, CA (US); Seema Rani BHAT, Santa Clara, CA (US)

(21) Appl. No.: 15/643,782

(22) Filed: Jul. 7, 2017

Related U.S. Application Data
(63) Continuation of application No. PCT/US2016/013069, filed on Jan. 12, 2016.
(60) Provisional application No. 62/102,540, filed on Jan. 12, 2015.

(51) Int. Cl. A61K 31/538; A61K 9/00

(52) U.S. Cl. CPC A61K 31/538; A61K 9/0053

(57) ABSTRACT

The present invention provides a method of using arylpiperazine derivatives for treating psychosis associated with Parkinson's disease. The method comprises a step of administering to a patient in need thereof an effective amount of a compound of Formula 1, which is an arylpiperazine derivative.

This application is a continuation of PCT/US2016/013069, filed Jan. 12, 2016; which claims the priority of U.S. Provisional Application No. 62/102,540, filed Jan. 12, 2015. The contents of the above-identified applications are incorporated herein by reference in their entireties.

### TECHNICAL FIELD

The present invention relates to methods of utilizing arylpiperazine derivatives for treating psychosis associated with Parkinson's disease.

### BACKGROUND

Psychotic symptoms are a common feature of Parkinson's disease (PD) and are related to disease duration and severity, dementia, depression, and age. The pathophysiology of psychosis is now known to involve an interaction between extrinsic, drug-related and intrinsic, disease-related components such as neurochemical (dopamine, serotonin, acetycholine, etc) and structural abnormalities, visual processing deficits, sleep dysregulation, and genetics (Wint et al., 2004; Zahodne and Fernandez 2008). Although effective in addressing motor dysfunction, long-term use of anti-Parkinsonian agents has been implicated as a component in the development of psychiatric side effects including psychosis which occurs in about 50% of patients. It is generally accepted that the most effective first-line strategy in the treatment of psychosis in Parkinson's disease is a reduction in anti-Parkinson's disease medications. If the reduction in anti-PD medications to the lowest dose tolerable without the exacerbation of motor symptoms does not improve psychosis, the addition of an antipsychotic agent is considered. Often use of atypical antipsychotics have multiple adverse effects such as extrapyramidal symptoms, metabolic and cardiac effects, but also negative cognitive effects.

Thus, there is a need for more effective therapies for treating psychotic symptoms in Parkinson's disease.

### DETAILED DESCRIPTION OF THE INVENTION

### Definitions

“Alkyl” or “alkanyl” refers to a saturated, branched or straight-chain or cyclic monovalent hydrocarbon radical derived by the removal of one hydrogen atom from a single carbon atom of a parent alkane. Typical alkyl groups include, but are not limited to methyl; ethyl; propyls such as propan-1-yl, propan-2yl, cyclopropan-1-yl; butyls such as butan-1-yl, butan-2-yl, 2-methyl-propan-1-yl, 2-methyl-propan-2-yl, cyclobutan-1-yland the like. Preferably, an alkyl group comprises from 1-20 carbon atoms, more preferably, from 1 to 10, or 1 to 6, or 1-4 carbon atoms.

“Alkenyl” refers to an unsaturated branched, straight-chain or cyclic alkyl radical having at least one carbon-carbon double bond derived by the removal of one hydrogen atom from a single carbon atom of a parent alkene. The group may be in either the cis or trans conformation about the double bond(s). Typical alkenyl groups include, but are not limited to, ethenyl; propenyls such as prop-1-en-1-yl, prop-1-en-2-yl, prop-2-en-1-yl (allyl), prop-2-en-2-yl, cycloprop-1-en-1-yl, cycloprop-2-en-1-yl; butenyls such as but-1-en-1-yl, but-1-en-2-yl, 2-methy-prop-1-en-1-yl, but-2-en-1-yl, but-2-en-2-yl, buta-1,3-dien-1-yl, buta-1,3-dien-2-yl, cyclobut-1-en-1-yl, cyclobut-1-en-3-yl, cyclobuta-1,3-dien 1-yl, etc.; and the like.

“Alkynyl” refers to an unsaturated branched, straight-chain or cyclic alkyl radical having at least one carbon-carbon triple bond derived by the removal of one hydrogen atom from a single carbon atom of a parent alkyne. Typical alkynyl groups include, but are not limited to, ethynyl; propynyls such as prop-1-yn-1-yl, prop-2-yn-1-yl, etc.; butynyls such as but-1-yn-1-yl, but-1-yn3-yl, but-3-yn-1-yl, etc.; and the like.

“Acyl” refers to a radical —C(O)R, where R is hydrogen, alkyl, cycloalkyl, cycloheteroalkyl, aryl, arylalkyl, heteroalkyl, heteroaryl, heteroarylalkyl, as defined herein that may be optionally substituted by one or more substituents as defined herein. Representative examples include, but are not limited to formyl, acetyl, cyclohexylcarbonyl, cyclohexylmethylcarbonyl, benzoyl, benzylcarbonyl and the like.

“Acyloxyalkyloxycarbonyl” refers to a radical —C(O)OCR′R″OC(O)R′″, where R′, R″, and R″′ are each independently hydrogen, alkyl, cycloalkyl, cycloheteroalkyl, aryl, arylalkyl, heteroalkyl, heteroaryl, heteroarylalkyl, as defined herein that may be optionally substituted by one or more substituents as defined herein. Representative examples include, but not limited to —C(O)OCH₂OC(O)CH₃, —C(O)OCH₂OC(O)CH₂CH₃, —C(O)OCH(CH₃)OC(O)CH₂CH₃, —C(O)OCH(CH₃)OC(O)C₆H₅ and the like.

“Acylalkyloxycarbonyl” refers to a radical —C(O)OCR′R″C(O)R′″, where R′, R″, and R″ are each independently hydrogen, alkyl, cycloalkyl, cycloheteroalkyl, aryl, arylalkyl, heteroalkyl, heteroaryl, heteroarylalkyl, as defined herein that may be optionally substituted by one or more substituents as defined herein. Representative examples include, but not limited to —C(O)OCH₂C(O)CH₃, —C(O)OCH₂C(O)CH₂CH₃, —C(O)OCH(CH₃)C(O)CH₂CH₃, —C(O)OCH(CH₃)C(O)C₆H₅ and the like.

“Acyloxyalkyloxycarbonylamino” refers to a radical —NRC(O)OCR′R″OC(O)R′″, where R, R′, R″, and R′″ are each independently hydrogen, alkyl, cycloalkyl, cycloheteroalkyl, aryl, arylalkyl, heteroalkyl, heteroaryl, heteroarylalkyl, as defined herein that may be optionally substituted by one or more substituents as defined herein. Representative examples include, but not limited to —NHC(O)OCH₂OC(O)CH₃, —NHC(O)OCH₂OC(O)CH₂CH₃, —NHC(O)OCH(CH₃)OC(O)CH₂CH₃, —NHC(O)OCH(CH₃)OC(O)C₆H₅ and the like.

“Acylalkyloxycarbonylamino” refers to a radical —NRC(O)OCR′R″C(O)R′″, where R, R′, R″, and R′″ are each independently hydrogen, alkyl, cycloalkyl, cycloheteroalkyl, aryl, arylalkyl, heteroalkyl, heteroaryl, heteroarylalkyl, as defined herein that may be optionally substituted by one or more substituents as defined herein. Representative examples include, but not limited to —NHC(O)OCH₂C(O)CH₃, —NHC(O)OCH₂C(O)CH₂CH₃, —NHC(O)OCH(CH₃)C(O)CH₂CH₃, —NHC(O)OCH(CH₃)C(O)C₆H₅ and the like.

“Acylamino” refers to “amide” as defined herein.

“Alkylamino” means a radical —NHR where R represents an alkyl, or cycloalkyl group as defined herein that may be optionally substituted by one or more substituents as defined herein. Representative examples include, but are not limited to, methylamino, ethylamino, 1-methylethylamino, cyclohexylamino and the like.

“Alkoxy” refers to a radical —OR where R represents an alkyl, or cycloalkyl group as defined herein that may be optionally substituted by one or more substituents as defined herein. Representative examples include, but are not limited to methoxy, ethoxy, propoxy, butoxy, cyclohexyloxy and the like.

“Alkoxycarbonyl” refers to a radical —C(O)-alkoxy where alkoxy is as defined herein.

“Alkoxycarbonylalkoxy” refers to a radical —OCR′R″C(O)-alkoxy where alkoxy is as defined herein. Similarly, where R′ and R″ are each independently hydrogen, alkyl, cycloalkyl, cycloheteroalkyl, aryl, arylalkyl, heteroalkyl, heteroaryl, heteroarylalkyl, as defined herein that may be optionally substituted by one or more substituents as defined herein. Representative examples include, but are not limited to —OCH₂C(O)OCH₃, —OCH₂C(O)OCH₂CH₃, —OCH(CH₃)C(O)OCH₂CH₃, —OCH(C₆H₅)C(O)OCH₂CH₃, —OCH(CH₂C₆H₅)C(O)OCH₂CH₃, —OC(CH₃)(CH₃)C(O)OCH₂CH₃, and the like.

“Alkoxycarbonylalkylamino” refers to a radical —NRCR′R″C(O)-alkoxy where alkoxy is as defined herein. Similarly, where R, R′, R′ and R″ are each independently hydrogen, alkyl, cycloalkyl, cycloheteroalkyl, aryl, arylalkyl, heteroalkyl, heteroaryl, heteroarylalkyl, as defined herein that may be optionally substituted by one or more substituents as defined herein. Representative examples include, but are not limited to —NHCH₂C(O)OCH₃, —N(CH₃)CH₂C(O)OCH₂CH₃, —NHCH(CH₃)C(O)OCH₂CH₃, —NHCH(C₆H₅)C(O)OCH₂CH₃, —NHCH(CH₂C₆H₅)C(O)OCH₂CH₃, —NHC(CH₃)(CH₃)C(O)OCH₂CH₃, and the like.

“Alkylsulfonyl” refers to a radical —S(O)₂R where R is an alkyl, or cycloalkyl group as defined herein that may be optionally substituted by one or more substituents as defined herein. Representative examples include, but are not limited to, methylsulfonyl, ethylsulfonyl, propylsulfonyl, butylsulfonyl, and the like.

“Alkylsulfinyl” refers to a radical —S(O)R where R is an alkyl, or cycloalkyl group as defined herein that may be optionally substituted by one or more substituents as defined herein. Representative examples include, but are not limited to, methylsulfinyl, ethylsulfinyl, propylsulfinyl, butylsulfinyl, and the like.

“Alkylthio” refers to a radical —SR where R is an alkyl or cycloalkyl group as defined/herein that may be optionally substituted by one or more substituents as defined herein. Representative examples include, but are not limited to methylthio, ethylthio, propylthio, butylthio, and the like.

“Amide” or “acylamino” refers to a radical —NR′C(O)R″, where R′ and R″ are each independently hydrogen, alkyl, cycloalkyl, cycloheteroalkyl, aryl, arylalkyl, heteroalkyl, heteroaryl, heteroarylalkyl, as defined herein that may be optionally substituted by one or more substituents as defined herein. Representative examples include, but are not limited to, formylamino acetylamino, cyclohexylcarbonylamino, cyclohexylmethylcarbonyl-amino, benzoylamino, benzylcarbonylamino and the like.

“Aryl” refers to a monovalent aromatic hydrocarbon radical derived by the removal of one hydrogen atom from a single carbon atom of a parent aromatic ring system. Typical aryl groups include, but are not limited to, groups derived from aceanthrylene, acenaphthylene, acephenanthrylene, anthracene, azulene, benzene, chrysene, coronene, fluoranthene, fluorine, hexacene, hexaphene, hexalene, as-indacene, s-indacene, indane, indene, naphthalene, octacene, octaphene, octalene, ovalene, penta-2,4-diene, pentacene, pentalene, pentaphene, perylene, phenalene, phenanthrene, picene, pleidene, pyrene, pyranthrene, rubicene, triphenylene, trinaphthalene, and the like. Preferable, an aryl group comprises from 6 to 20 carbon atoms, more preferably, between 6 to 12 carbon atoms.

“Arylalkyl” refers to an acyclic alkyl in which one of the hydrogen atoms bonded to a carbon atom, typically a terminal or sp³ carbon atom, is replaced with an aryl group. Typically arylalkyl groups include, but not limited to, benzyl, 2-phenylethan-1-yl, naphthylmethyl, 2-naphthylethan-1-yl, naphthobenzyl, 2-naphthophenylethan-1-yl and the like. Preferably, an arylalkyl group is (C₆-C₃)arylalkyl, e.g., the alkyl moiety of the arylalkyl group is (C₁-C₁₀) and the aryl moiety is (C₆-C₂₀), more preferably, an arylalkyl group is (C₆-C₂₀) arylalkyl, e.g., the alkyl moiety of the arylalkyl group is (C₁-C₈) and the aryl moiety is (C₆-C₁₂).

“Arylalkoxy” refers to an —O-arylalkyl radical where arylalkyl is as defined herein that may be optionally substituted by one or more substituents as defined herein.

“Arylalkoxycarbonylalkoxy” refers to a radical —OCR′R″C(O)-arylalkoxy where arylalkoxy is as defined herein. Similarly, where R′ and R″ are each independently hydrogen, alkyl, cycloalkyl, cycloheteroalkyl, aryl, arylalkyl, heteroalkyl, heteroaryl, heteroarylalkyl, as defined herein that may be optionally substituted by one or more substituents as defined herein. Representative examples include, but are not limited to —OCH₂C(O)OCH₂C₆H₅, —OCH(CH₃)C(O)O CH₂C₆H₅, —OCH(C₆H₅)C(O)O CH₂C₆H₅, —OCH(CH₂C₆H₅)C(O)O CH₂C₆H₅, —OC(CH₃)(CH₃)C(O)O CH₂C₆H₅, and the like.

“Arylalkoxycarbonylalkylamino” refers to a radical —NRCR′R″C(O)-arylalkoxy where arylalkoxy is as defined herein. Similarly, where R, R′, R′ and R″ are each independently hydrogen, alkyl, cycloalkyl, cycloheteroalkyl, aryl, arylalkyl, heteroalkyl, heteroaryl, heteroarylalkyl, as defined herein that may be optionally substituted by one or more substituents as defined herein. Representative examples include, but are not limited to —NHCH₂C(O)OCH₂C₆H₅, —N(CH₃)CH₂C(O)OCH₂C₆H₅, —NHCH(CH₃)C(O)OCH₂C₆H₅, —NHCH(C₆H₅)C(O)OCH₂C₆H₅, —NHCH(CH₂C₆H₅)C(O)OCH₂C₆H₅, —NHC(CH₃)(CH₃)C(O)OCH₂C₆H₅, and the like.

“Aryloxycarbonyl” refers to radical —C(O)—O-aryl where aryl is defined herein that may be optionally substituted by one or more substituents as defined herein.

“Aryloxycarbonylalkoxy” refers to a radical —OCR′R″C(O)-aryloxy where aryloxy is as defined herein. Similarly, where R′ and R″ are each independently hydrogen, alkyl, cycloalkyl, cycloheteroalkyl, aryl, arylalkyl, heteroalkyl, heteroaryl, heteroarylalkyl, as defined herein that may be optionally substituted by one or more substituents as defined herein. Representative examples include, but are not limited to —OCH₂C(O)OC₆H₅, —OCH(CH₃)C(O)OC₆H₅, —OCH(C₆H₅)C(O)OC₆H₅, —OCH(CH₂C₆H₅)C(O)OC₆H₅, —OC(CH₃)(CH₃)C(O)OC₆H₅, and the like.

“Aryloxycarbonylalkylamino” refers to a radical —NRCR′R″C(O)-aryloxy where aryloxy is as defined herein. Similarly, where R, R′, R′ and R″ are each independently hydrogen, alkyl, cycloalkyl, cycloheteroalkyl, aryl, arylalkyl, heteroalkyl, heteroaryl, heteroarylalkyl, as defined herein that may be optionally substituted by one or more substituents as defined herein. Representative examples include, but are not limited to —NHCH₂C(O)OC₆H₅, —N(CH₃)CH₂C(O)OC₆H₅, —NHCH(CH₃)C(O)OC₆H₅, —NHCH(C₆H₅)C(O)OC₆H₅, —NHCH(CH₂C₆H₅)C(O)OC₆H₅, —NHC(CH₃)(CH₃)C(O)OC₆H₅, and the like.

“Carbamoyl” refers to the radical —C(O)NRR where each R group is independently, hydrogen, alkyl, cycloalkyl, cycloheteroalkyl, aryl, arylalkyl, heteroalkyl, heteroaryl, heteroarylalkyl, as defined herein that may be optionally substituted by one or more substituents as defined herein.

“Carbamate” refers to a radical —NR′C(O)OR″, where R′ and R″ are each independently hydrogen, alkyl, cycloalkyl, cycloheteroalkyl, aryl, arylalkyl, heteroalkyl, heteroaryl, heteroarylalkyl, as defined herein that may be optionally substituted by one or more substituents as defined herein. Representative examples include, but are not limited to, methylcarbamate (—NHC(O)OCH₃), ethylcarbamate (—NHC(O)OCH₂CH₃), benzylcarbamate (—NHC(O)OCH₂C₆H₅), and the like.

“Carbonate” refers to a radical —OC(O)OR, where R is alkyl, cycloalkyl, cycloheteroalkyl, aryl, arylalkyl, heteroalkyl, heteroaryl, heteroarylalkyl, as defined herein that may be optionally substituted by one or more substituents as defined herein. Representative examples include, but are not limited to, methyl carbonate (—C(O)OCH₃), cyclohexyl carbonate (—C(O)OC₆H₁₁), phenyl carbonate (—C(O)OC₆H₅), benzyl carbonate (—C(O)OCH₂C₆H₅), and the like.

“Cycloalkyl” refers to a substituted or unsubstituted cylic alkyl radical. Typical cycloalkyl groups include, but are not limited to, groups derived from cyclopropane, cyclobutane, cyclopentane, cyclohexane, and the like. In a preferred embodiment, the cycloalkyl group is (C₃-C₁₀) cycloalkyl, more preferably (C₃-C₇) cycloalkyl.

“Cycloheteroalkyl” refers to a saturated or unsaturated cyclic alkyl radical in which one or more carbon atoms (and any associated hydrogen atoms) are independently replaced with the same or different heteroatom. Typical heteroatoms to replace the carbon atom(s) include, but are not limited to, N, P, O, S, Si, etc. Where a specific level of saturation is intended, the nomenclature “cycloheteroalkanyl” or “cycloheteroalkenyl” is used. Typical cycloheteroalkyl groups include, but are not limited to, groups derived from epoxides, imidazolidine, morpholine, piperazine, piperidine, pyrazolidine, pyrrolidine, quinuclidine, and the like.

“Cycloheteroalkoxycarbonyl” refers to a radical —C(O)—OR where R is cycloheteroalkyl as defined herein that may be optionally substituted by one or more substituents as defined herein.

“Dialkylamino” means a radical —NRR′ where R and R′ independently represent an alkyl or cycloalkyl group as defined herein that may be optionally substituted by one or more substituents as defined herein. Representative examples include, but are not limited to dimethylamino, methylethylamino, di-(1-methylethyl)amino, (cyclohexyl)(methyl)amino, (cyclohexyl)(ethyl)amino, (cyclohexyl)(propyl)amino, and the like.

“Ester” refers to a radical —C(O)OR, where R is alkyl, substituted alkyl, cycloalkyl, substituted cycloalkyl, cycloheteroalkyl, substituted cycloheteroalkyl, aryl, substituted aryl, arylalkyl, substituted arylalkyl, heteroalkyl, substituted heteroalkyl, heteroaryl, substituted heteroaryl, heteroarylalkyl, substituted heteroarylalkyl as defined herein that may be optionally substituted by one or more substituents as defined herein. Representative examples include, but are not limited to, methyl ester (—C(O)OCH₃), cyclohexyl ester (—C(O)OC₆H₁₁), phenyl ester (—C(O)OC₆H₅), benzyl ester (—C(O)OCH₂C₆H₅), and the like.

“Ether” refers to a radical —OR, where R is alkyl, cycloalkyl, cycloheteroalkyl, aryl, arylalkyl, heteroalkyl, heteroaryl, heteroarylalkyl, as defined herein that may be optionally substituted by one or more substituents as defined herein.

“Halogen” means fluoro, chloro, bromo, or iodo.

“Heteroaryl” refers to a monovalent heteroaromatic radical derived by the removal of one hydrogen atom from a single atom of a parent heteroaromatic ring system. Typical heteroaryl groups include, but are not limited to, groups derived from acridine, arsindole, carbazole, carboline, chromane, chromene, cinnoline, furan, imidazole, indazole, indole, indoline, indolizine, isobenzofuran, isochromene, isoindole, isoindoline, isoquinoline, isothiazole, isoxazole, naphthyridine, oxadiazole, oxazole, perimidine, phenanthridine, phenanthroline, phenazine, phthalazine, pteridine, purine, pyran, pyrazine, pyrazole, pyridazine, pyridine, pyrimidine, pyrrole, pyrrolizine, quinazoline, quinoline, quinolizine, quinoxaline, tetrazole, thiadiazole, thiazole, thiophene, triazole, xanthene, and the like. Preferably, the heteroaryl group is between 5-20 membered heteroaryl, with 5-10 membered heteroaryl being particularly preferred. Preferred heteroaryl groups are those derived from thiophene, pyrrole, benzothiophene, benzofuran, indole, pyridine, quinoline, imidazole, oxazole and pyrazine.

“Heteroaryloxycarbonyl” refers to a radical —C(O)—OR where R is heteroaryl as defined that may be optionally substituted by one or more substituents as defined herein.

“Heteroarylalkyl” refers to an acyclic alkyl radical in which one of the hydrogen atoms bonded to a carbon atom, typically a terminal or sp3 carbon atom, is replaced with a heteroaryl group. Preferably, the heteroarylalkyl radical is a 6-30 carbon membered heteroarylalkyl, e.g., the alkyl moiety of the heteroarylalkyl is 1-10 membered and the heteroaryl moiety is a 5-20 membered heteroaryl, more preferably, a 6-20 membered heteroarylalkyl, e.g., the alkyl moiety of the heteroarylalkyl is 1-8 membered and the heteroaryl moiety is a 5-12 membered heteroaryl.

“Oxo” means the divalent radical ═O.

“Pharmaceutically acceptable” means approved or approvable by a regulatory agency of the Federal or state government or listed in the U.S. Pharmacopoeia or other generally recognized pharmacopoeia for use in animals, and more particularly in humans.

“Pharmaceutically acceptable salt” refers to a salt of a compound of the invention, which is pharmaceutically acceptable and possesses the desired pharmacological activity of the parent compound. Such salts include: (1) acid addition salts, formed with inorganic acids such as hydrochloric acid, hydrobromic acid, sulfuric acid, nitric acid, phosphoric acid, and the like; or formed with organic acids such as acetic acid, propionic acid, hexanoic acid, cyclopentane propionic acid, glycolic acid, pyruvic acid, lactic acid, malonic acid, succinic acid, malic acid, maleic acid, fumaric acid, tartaric acid, citric acid, benzoic acid, 3-(4-hydroxybenzoyl)benzoic acid, cinnamic acid, mandelic acid, methanesulfonic acid, ethanesulfonic acid, 1,2-ethanedisulfonic acid, 2-hydroxyethanesulfonic acid, benzenesulfonic acid, 4-chlorobenzenesulfonic acid, 2-naphthalenesulfonic acid, 4-toluenesulfonic acid, camphorsulfonic acid, 4-methylbicyclo[2,2,2]-oct-2-ene-1-carboxylic acid, glucoheptonic acid, 3-phenylpropionic acid, trimethylacetic acid, tertiary butylacetic acid, laurylsulfuric acid, gluconic acid, glutamic acid, hydroxynaphthoic acid, salicylic acid, stearic acid, muconic acid, and the like; or (2) salts formed when an acidic proton present in the parent compound is replaced by a metal ion, e.g., an alkali metal ion, an alkaline earth ion, or an aluminum ion; or coordinates with an organic base such as ethanolamine, diethanolamine, triethanolamine, N-methylglucamine and the like.

“Pharmaceutically acceptable vehicle” refers to a diluent, adjuvant, excipient or carrier with which a compound of the invention is administered.

“Phosphate” refers to a radical —OP(O)(OR′)(OR″), where R′ and R″ are each independently hydrogen, alkyl, cycloalkyl, cycloheteroalkyl, aryl, arylalkyl, heteroalkyl, heteroaryl, heteroarylalkyl, as defined herein that may be optionally substituted by one or more substituents as defined herein.

“Phosphonate” refers to a radical —P(O)(OR′)(OR″), where R′ and R″ are each independently hydrogen, alkyl, cycloalkyl, cycloheteroalkyl, aryl, arylalkyl, heteroalkyl, heteroaryl, heteroarylalkyl, as defined herein that may be optionally substituted by one or more substituents as defined herein.

“Racemate” refers to an equimolar mixture of enantiomers of a chiral molecule.

“Substituted” refers to a group in which one or more hydrogen atoms are each independently replaced with the same or different substituents(s). Typical substituents include, but are not limited to , —X, —R⁵⁴, —O⁻, =O, —OR⁵⁴, —SR⁵⁴, —S, ═S, —NR⁵⁴R⁵⁵, ═NR⁵⁴, —CX₃, —CF₃, —CN, —OCN, —SCN, —NO, —NO₂, =N₂, —N₃, —S(O)₂O—, —S(O)₂OH, —S(O)₂OR⁵⁴, —OS(O)₂O³¹, —OS(O)₂R⁵⁴, —P(O)(O—)₂, —P(O)(OR¹4)(O³¹), —OP(O)(OR⁵⁴)(OR⁵⁵), —C(O)R⁵⁴, —C(S)R⁵⁴, —C(O)OR⁵⁴, —C(O)NR⁵⁴R⁵⁵, —C(O)O—, —C(S)OR⁵⁴, —NR⁵⁶C(O)NR⁵⁴R⁵⁵, —NR⁵⁶C(S)NR⁵⁴R⁵⁵, —NR⁵⁷C(NR⁵⁶)NR⁵⁴R⁵⁵, and —C(NR⁵⁶)NR⁵⁴R⁵⁵, where each X is independently a halogen; each R⁵⁴, R⁵⁵, R⁵⁶ and R⁵⁷ are independently hydrogen, alkyl, substituted alkyl, aryl, substituted aryl, arylalkyl, substituted arylalkyl, cycloalkyl, substituted cycloalkyl, cycloheteroalkyl, substituted cycloheteroalkyl, heteroalkyl, substituted heteroalkyl, heteroaryl, substituted heteroaryl, heteroarylalkyl, substituted heteroarylalkyl, —NR⁵⁸R⁵⁹, —C(O)R⁵⁸ or —S(O)₂R⁵⁸ or optionally R⁵⁸ and R⁵⁹ together with the atom to which they are both attached form a cycloheteroalkyl or substituted cycloheteroalkyl ring; and R⁵⁸ and R⁵⁹ are independently hydrogen, alkyl, substituted alkyl, aryl, substituted aryl, arylalkyl, substituted arylalkyl, cycloalkyl, substituted cycloalkyl, cycloheteroalkyl, substituted cycloheteroalkyl, heteroalkyl, substituted heteroalkyl, heteroaryl, substituted heteroaryl, heteroarylalkyl, substituted heteroarylalkyl.

“Sulfate” refers to a radical —OS(O)(O)OR, where R is hydrogen, alkyl, cycloalkyl, cycloheteroalkyl, aryl, arylalkyl, heteroalkyl, heteroaryl, heteroarylalkyl, as defined herein that may be optionally substituted by one or more substituents as defined herein.

“Sulfonamide” refers to a radical —S(O)(O)NR′R″, where R′ and R″ are independently hydrogen, alkyl, cycloalkyl, cycloheteroalkyl, aryl, arylalkyl, heteroalkyl, heteroaryl, heteroarylalkyl, as defined herein that may be optionally substituted by one or more substituents as defined herein or optionally R′ and R″ together with the atom to which they are both attached form a cycloheteroalkyl or substituted cycloheteroalkyl ring. Representative examples include but not limited to azetidinyl, pyrrolidinyl, piperidinyl, morpholinyl, 4-(NR′″)-piperazinyl or imidazolyl group wherein said group may be optionally substituted by one or more substituents as defined herein. R′″ hydrogen, alkyl, cycloalkyl, cycloheteroalkyl, aryl, arylalkyl, heteroalkyl, heteroaryl, heteroarylalkyl, as defined herein that may be optionally substituted by one or more substituents as defined herein.

“Sulfonate” refers to a radical —S(O)(O)OR, where R is hydrogen, alkyl, cycloalkyl, cycloheteroalkyl, aryl, arylalkyl, heteroalkyl, heteroaryl, heteroarylalkyl, as defined herein that may be optionally substituted by one or more substituents as defined herein.

“Thio” means the radical —SH.

“Thioether” refers to a radical —SR, where R is alkyl, cycloalkyl, cycloheteroalkyl, aryl, arylalkyl, heteroalkyl, heteroaryl, heteroarylalkyl, as defined herein that may be optionally substituted by one or more substituents as defined herein.

“Treating” or “Treatment” of any disease or disorder refers, in one embodiment, to ameliorating the disease or disorder (i.e., arresting or reducing the development of the disease or at least one of the clinical symptoms thereof). In another embodiment “treating” or “treatment” refers to ameliorating at least one physical parameter, which may not be discernible by the patient. In yet another embodiment, “treating” or “treatment” refers to inhibiting the disease or disorder, either physically (e.g., stabilization of a discernible symptom), physiologically, (e.g., stabilization of a physical parameter), or both.

“Therapeutically effective amount” means the amount of a compound that, when administered to a patient for treating a disease, is sufficient to effect such treatment for the disease. The “therapeutically effective amount” will vary depending on the compound, the disease and is severity and the age, weight, etc., of the patient to be treated, and can be determined by one of skill in the art without undue experimentation.

The present invention is directed to a method for treating psychotic symptoms in Parkinson's Disease.

### Compounds Useful in the Invention

Compounds of Formula (I) are useful for the present invention:

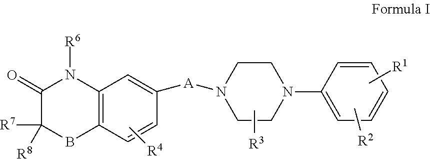

wherein:
A is —(CH₂)ₙ—, O—(CH₂)ₙ—, —S—(CH₂)ₙ—, —S(O)(O)—(CH₂)ₙ—, —NH—(CH₂)ₙ—, —CH₂—O—(CH₂)ₙ—, —(CH₂)ₙ—O—CH₂—CH₂—, —CH₂—S—(CH₂)ₙ—, —(CH₂)ₙ—S—CH₂—CH₂—, —CH₂—S(O)(O)—(CH₂)ₙ—, —(CH₂)ₙ, —S(O)(O)—CH₂—CH₂—, —O—C(O)—(CH₂)ₙ—, —S—C(O)—(CH₂)ₙ—, —NH—C(O)—(CH₂)ₙ—, —CH₂—C(O)—O—(CH₂)ₙ—, —CH₂—C(O)—NH—(CH₂)ₙ—, —CH₂—C(O)—S—(CH₂)ₙ—, —(CH₂)ₙ—C(O)—O—CH₂—CH₂—, —(CH₂)ₙ—C(O)—NH—CH₂—CH₂—, —(CH₂)ₙ—C(O)—S—CH₂—CH₂—, —CH₂—O—C(O)—(CH₂)ₙ—, —CH₂—NH—C(O)—(CH₂)ₙ—, —CH₂—S—C(O)—(CH₂)ₙ—, —(CH₂)ₙ—O—C(O)—CH₂—CH₂—, (CH₂)ₙ—NH—C(O)—CH₂—CH₂—, or (CH₂)ₙ—S—C(O)—CH₂—CH₂—, wherein n is an integer from 1 to 7, preferably n is 2 to 5, for example n is 4;

### B is O, S, S(O)(O), or NR⁵; and

each of R¹, R², R³, R⁴, R⁵, R⁶, R⁷, and R⁸ is independently hydrogen, alkyl, substituted alkyl, aryl, substituted aryl, arylalkyl, substituted arylalkyl, cycloalkyl, substituted cycloalkyl, cycloheteroalkyl, substituted cycloheteroalkyl, heteroaryl, substituted heteroaryl, heteroarylalkyl, substituted heteroarylalkyl, acylalkyloxycarbonyl, acyloxyalkyloxycarbonyl, acylalkyloxycarbonylamino, acyloxyalkyloxycarbonylamino, alkoxy, alkoxycarbonyl, alkoxycarbonylalkoxy, alkoxycarbonyllalkylamino, alkylsulfinyl, alkylsulfonyl, alkylthio, amino, alkylamino, arylalkylamino, dialkylamino, arylalkoxy, arylalkoxycarbonylalkoxy, arylalkoxycarbonylalkylamino, aryloxycarbonyl, aryloxycarbonylalkoxy, aryloxycarbonylalkylamino, carboxy, carbamoyl, carbamate, carbonate, cyano, halo, heteroaryloxycarbonyl, hydroxy, phosphate, phosphonate, sulfate, sulfonate, or sulfonamide, wherein R¹, R², R³, R⁴, R⁵, R⁶, R⁷ and R⁸ and A may optionally be substituted with isotopes that include, but not limited to ²H (deuterium), ³H (tritium), ¹³C, ³⁶Cl, ¹⁸F, ¹⁵N, ¹⁷O, ¹⁸O, ³¹P, ³²P, and ³⁵S; with ²H (deuterium) being preferred;
or a pharmaceutically acceptable salt, racemate or diastereomeric mixtures thereof.

In one aspect of the invention, A is —(CH₂)ₙ—.

In another aspect of the invention, A is —O—(CH₂)ₙ—, —S—(CH₂)ₙ—, —CH₂—O—(CH₂)ₙ—, —(CH₂)—O—CH₂—CH₂—, —CH₂—S—(CH₂)ₙ—, or —(CH₂)—S—CH₂—CH₂—; with A being O—(CH₂)ₙ— such as —O—(CH₂)₄— preferred.

In another aspect of the invention, A is —NH—C(O)—(CH₂)ₙ—, —CH₂—NH—C(O)—(CH₂)ₙ, —CH₂—C(O)—NH—(CH₂)ₙ— or —(CH₂)ₙ—C(O)—NH—CH₂—CH₂—.

In another aspect of the invention, B is O.

In another aspect of the invention, R³, R⁴, R⁶, R⁶, and R⁸ are H.

In another aspect of the invention, each of R¹ and R² is independently H, halogen (e.g., chloro), haloalkyl, or alkoxy (e.g., methoxy or ethoxy); preferably halogen or alkoxy.

In a preferred embodiment, A is O—(CH₂)ₙ—, n=2-5; B is O; R³, R⁴, R⁶, R⁶, and R⁸ are H; and R¹ and R² is independently H, halogen, haloalkyl, or alkoxy.

Preferred compounds of Formula I include, for example,

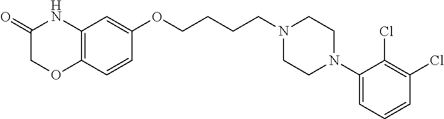 [SMILES: O=C1COc2ccc(OCCCCN3CCN(c4cccc(Cl)c4Cl)CC3)cc2N1]

6-(4-(4-(2,3-dichlorophenyl)piperazin-1-yl)butoxy)-2H-benzo[b][1,4]oxazin-3(-4H)-one, and its hydrochloride salt (Compound A); and

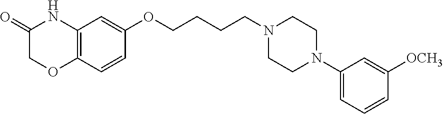 [SMILES: COc1cccc(N2CCN(CCCCOc3ccc4c(c3)NC(=O)CO4)CC2)c1]

6-(4-(4-(2-methoxyphenyl)piperazin-1-yl)butoxy)-2H-benzo[b][1,4]oxazin-3(-4H)-one, and its hydrochloride salt (Compound B).

The compounds useful for the present invention further pertain to enantiomerically isolated compounds of Formula I. The isolated enantiomeric forms of the compounds of Formula I are substantially free from one another (i.e., in enantiomeric excess). In other words, the “R” forms of the compounds are substantially free from the “S” forms of the compounds and are, thus, in enantiomeric excess of the “S” forms. Conversely, “S” forms of the compounds are substantially free of “R” forms of the compounds and are, thus, in enantiomeric excess of the “R” forms. In one embodiment of the invention, the isolated enantiomeric compounds are at least about in 80% enantiomeric excess. Thus, for example, the compounds are at least about 90% enantiomeric excess, preferably at least about 95% enantiomeric excess, more preferably at least about 97% enantiomeric excess., or even more preferably, at least 99% or greater than 99% enantiomeric excess.

Formula I compounds can be synthesized according U.S. Pat. No. 8,188,076, which is incorporated herewith in its entirety.

### Method of Treating Psychosis Associated with Parkinson's Disease

The present invention is directed to a method for treating psychosis associated with Parkinson's disease. The method comprises the step of administering an effective amount of a compound of Formula I to a patient in need thereof. Patients typically have a clinical diagnosis of idiopathic Parkinson's Disease, defined as the presence of at least three of the following cardinal features, in the absence of alternative explanations or atypical features: rest tremor, rigidity, Bradykinesia and/or akinesia, and postural and gait abnormalities. Patients also have symptoms of psychosis with presence of visual and/or auditory hallucinations, with or without delusion.

The present invention is effective for treating psychosis associated with Parkinson's disease. Psychotic symptoms in Parkinson's disease are caused by imbalance of dopamine-serotonin systems in the brain. Compounds of Formula I have potent binding affinity at the serotonin 5-HT2A receptor (compound B, Ki=2.5 nM, see Example 1) and 5-HT2B receptor (compound B, Ki=0.19 nM, see Example 1). In addition, compounds of Formula I exhibit partial agonist activities for the key subtypes of dopamine (D1, D2, D3 and D4) and serotonin (5-HT1A), and antagonist activity at the serotonin 5-HT6 and 5-HT7 receptors. Compounds of Formula I are potent dopamine and serotonin system modulators due to their selectivity, potent binding affinities, and especially partial agonist activities for key dopamine and serotonin receptors. Due to the unique interaction of compounds of Formula I to various dopamine and serotonin receptors, the inventors have discovered that Formula I compounds are effective for treating psychosis associated with Parkinson's disease.

In one embodiment, Formula I compounds treats memory impairment in patients having Parkinson's disease.

In one embodiment, Formula I compounds treats cognitive impairment in Parkinson's disease.

In one embodiment, Formula I compounds treats agitation in Parkinson's disease.

In one embodiment, Formula I compounds treats mood swing in Parkinson's disease.

In one embodiment, Formula I compounds treats psychosis in Parkinson's disease.

In one embodiment, Formula I compounds treats depression in Parkinson's disease.

Formula I compounds are effective in reducing the behavioral and psychological dysfunction in patients having Parkinson's disease. The treatment in general improves the primary outcomes such as Neuropsychiatric Inventory (NPI), function (Bristol Activities of Daily Living Scale, BADLS) and agitation (Cohen-Mansfield Agitation Inventory, CMAI). The treatment may also improve secondary outcomes such as Mini-Mental State Examination (MMSE), general functioning, caregiver burden and mortality. The evaluation of Psychosis in Parkinson's may use Unified Parkinson Disease Rating Scale and the Scale for Evaluation of Neuropsychiatric Disorders in Parkinson's disease (SEND-PD).

When used to treat psychosis in Parkinson's disease, one or more compounds of Formula I can be administered alone, or in combination with other agents, to a patient. The patient may be an animal, preferably a mammal, and more preferably a human.

Formula I compounds are preferably administered orally. Formula I compounds may also be administered by any other convenient route, for example, by infusion or bolus injection, by absorption through epithelial or mucocutaneous linings (e.g., oral mucosa, rectal and intestinal mucosa, etc.). Administration can be systemic or local. Various delivery systems are known, (e.g., encapsulation in liposomes, microparticles, microcapsules, capsules, etc.) that can be used to administer a compound and/or composition of the invention. Methods of administration include, but are not limited to, intradermal, intramuscular, intraperitoneal, intravenous, subcutaneous, intranasal, epidural, oral, sublingual, intranasal, intracerebral, intravabinal, transdermal, rectally, by inhalation, or topically, particularly to the ears, nose, eyes or skin. Transdermal administration may be preferred for young children.

Formula I compounds can be delivered via sustained release systems, preferably oral sustained release systems. In one embodiment, a pump may be used (see, Langer, supra; Sefton, 1987, CRC Crit. Ref Biomed. Eng. 14:201; Saudek et al., 1989, N. Engl. J. Med. 321:574).

In one embodiment, polymeric materials can be used (see “Medical Applications of Controlled Release,” Langer and Wise (eds.), Wiley, New York (1984); Ranger and Peppas, 1983, J. Macromol. Sci. Rev. Macromol Chem. 23:61; see also Levy et al., 1985, Science 228:190; During et al., 1989, Ann. Neurol. 25:351; Howard et al, 1989, J. Neurosurg. 71:105). In a preferred embodiment, polymeric materials are used for oral sustained release delivery. Preferred polymers include sodium carboxymethylcellulose, hydroxypropylcellulose, hydroxypropylmethylcellulose and hydroxyethylcellulose (most preferred, hydroxypropylmethylcellulose). Other preferred cellulose ethers have been described in the art (Bamba et al., Int. J. Pharm., 1979, 2, 307).

In one embodiment, enteric-coated preparations can be used for oral sustained release administration. Preferred coating materials include polymers with a pH-dependent solubility (i.e., pH-controlled release), polymers with a slow or pH-dependent rate of swelling, dissolution or erosion (i.e., time controlled release), polymers that are degraded by enzymes (i.e., enzyme controlled release) and polymers that form firm layers that are destroyed by an increase in pressure (i.e., pressure-controlled release).

In still another embodiment, osmotic delivery systems are used for oral sustained release administration (Verma et al., Drug Dev. Ind. Pharm., 2000, 26:695-708). In a preferred embodiment, OROS® osmotic delivery systems are used for oral sustained release delivery devices (See for example, Theeuwes et al., U.S. Pat. No. 3,845,770; and Theeuwes et al, U.S. Pat. No. 3,916,899).

In yet another embodiment, a controlled-release system can be placed in proximity of the target of the compounds and/or composition of the invention, thus requiring only a fraction of the systemic dose (See, e.g., Goodson, in “Medical Applications of Controlled Release,” supra, vol. 2, pp. 115-138(1984)). Other controlled-release systems discussed in Langer, 1990, Science 249:1527-1533 may also be used.

Formula I compounds may be cleaved either chemically and/or enzymatically. One or more enzymes present in the stomach, intestinal lumen, intestinal tissue, blood, liver, brain or any other suitable tissue of a mammal may enzymatically cleave the compounds and/or compositions of the invention.

### Pharmaceutical Formulation

The present invention is directed to a pharmaceutical formulation for treating psychosis associated with Parkinson's disease. The pharmaceutical formulation contains a therapeutically effective amount of one or more compounds of Formula I, preferably in purified form, together with a suitable amount of a pharmaceutically acceptable vehicle. When administered to a patient, the pharmaceutical formulation is preferably sterile. Water is a preferred vehicle when the compound of the invention is administered intravenously. Saline solutions and aqueous dextrose and glycerol solutions can also be employed as liquid vehicles, particularly for injectable solutions. Suitable pharmaceutical vehicles also include excipients such as starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like. The present agents, or pH buffering agents. In addition, auxiliary, stabilizing, thickening, lubricating and coloring agents may be used.

Pharmaceutical compositions comprising a compound of the invention may be manufactured by means of conventional mixing, dissolving, granulating, levigating, and emulsifying, encapsulating, entrapping or lyophilizing process. Pharmaceutical compositions may be formulated in conventional manner using one or more physiologically acceptable carriers, diluents, excipients or auxiliaries, which facilitate processing of compounds of the invention into preparations which can be used pharmaceutically. Proper formulation is dependent upon the route of administration chosen.

The present compositions can take the form of solutions, suspensions, emulsion, tablets, pills, pellets, and capsules, capsules containing liquids, powders, sustained-release formulations, suppositories, emulsions, aerosols, sprays, suspensions, or any other form suitable for use. In one embodiment, the pharmaceutically acceptable vehicle is a capsule (see e.g., Grosswald et al., U.S. Pat. No. 5,698,155). Other examples of suitable pharmaceutical vehicles have been described in the art (see Remington's Pharmaceutical Sciences, Philadelphia College of Pharmacy and Science, 17th Edition, 1985). Preferred compositions of the invention are formulated for oral delivery, particularly for oral sustained release administration.

Compositions for oral delivery may be in the form of tablets, lozenges, aqueous or oily suspensions, granules, powders, emulsions, capsules, syrups or elixirs, for example. Orally administered compositions may contain one or more optionally agents, for example, sweetening agents such as fructose, aspartame or saccharin; flavoring agents such as peppermint, oil of wintergreen, or cherry coloring agents and preserving agents to provide a pharmaceutically palatable preparation. Moreover, where in tablet or pill form, the compositions may be coated to delay disintegration and absorption in the gastrointestinal tract, thereby providing a sustained action over an extended period of time. Selectively permeable membranes surrounding an osmotically active driving compound are also suitable for orally administered compounds of the invention. In these later platforms, fluid from the environment surrounding the capsule is imbibed by the driving compound, which swells to displace the agent or agent composition through an aperture. These delivery platforms can provide an essentially zero order delivery profile as opposed to the spiked profiles of immediate release formulations. A time delay material such as glycerol monostearate or glycerol stearate may also be used. Oral compositions can include standard vehicles such as mannitol, lactose, starch, magnesium stearate, sodium saccharine, cellulose, magnesium carbonate, etc. Such vehicles are preferably of pharmaceutical grade.

For oral liquid preparations such as, for example, suspensions, elixirs and solutions, suitable carriers, excipients or diluents include water , saline, alkyleneglycols (e.g., propylene glycol), polyalkylene glycols (e.g., polyethylene glycol) oils, alcohols, slightly acidic buffers between pH 4 and pH 6 (e.g., acetate, citrate, ascorbate at between about mM to about 50 mM) etc. Additionally, flavoring agents, preservatives, coloring agents, bile salts, acylcarnitines and the like may be added.

Compositions for administration via other routes may also be contemplated. For buccal administration, the compositions may take the form of tablets, lozenges, etc. formulated in conventional manner. Liquid drug formulations suitable for use with nebulizers and liquid spray devices and EHD aerosol devices will typically include a compound of the invention with a pharmaceutically acceptable vehicle. Preferably, the pharmaceutically acceptable vehicle is a liquid such as alcohol, water, polyethylene glycol or a perfluorocarbon. Optionally, another material may be added to alter the aerosol properties of the solution or suspension of compounds of the invention. Preferably, this material is liquid such as alcohol, glycol, polyglycol or fatty acid. Other methods of formulating liquid drug solutions or suspension suitable for use in aerosol devices are known to those of skill in the art (see, e.g., Biesalski, U.S. Pat. No. 5,112,598; Biesalski, U.S. Pat. No. 5,556,611). A compound of the invention may also be formulated in rectal or vaginal compositions such as suppositories or retention enemas, e.g., containing conventional suppository bases such as cocoa, butter or other glycerides. In addition to the formulations described previously, a compound of the invention may also be formulated as depot preparation. Such long acting formulations may be administered by implantation (for example, subcutaneously or intramuscularly) or by intramuscular injection. Thus, for example, a compound of the invention may be formulated with suitable polymeric or hydrophobic materials (for example, as an emulsion in an acceptable oil) or ion exchange resins, or as sparingly soluble derivatives, for example, as a sparingly soluble salt.

### Dosage for the Treatment

The amount of Formula I compound administered is dependent on, among other factors, the subject being treated, and the weight of the subject, the severity of the affliction, the manner of administration and the judgment of the prescribing physician. For example, the dosage may be delivered in a pharmaceutical composition by a single administration, by multiple applications or controlled release. In one embodiment, the compounds of the invention are delivered by oral sustained release administration. In one embodiment, the compounds of the invention are administered twice per day, and preferably, once per day. Dosing may be repeated intermittently, may be provided alone or in combination with other drugs, and may continue as long as required for effective treatment of the disease state or disorder.

The compounds of Formula I may be administered in the range 0.1 mg to 500 mg, preferably 1 mg to 100 mg per day, such as 5 mg, 10 mg, 15 mg, 20 mg, 25 mg, 35 mg or 50 mg per day, and preferably 10 mg per day.

### Combination Therapy

In certain embodiments of the present invention, the compounds of the invention can be used in combination therapy with at least one other therapeutic agent. Formula I compounds and the therapeutic agent can act additively or synergistically. In one embodiment, Formula I compound is administered concurrently with the administration of another therapeutic agent, which can be part of the same composition of Formula I compound. In another embodiment, a composition comprising a compound of the invention is administered prior or subsequent to administration of another therapeutic agent.

The invention is further illustrated by the following examples.

### EXAMPLES

### Example 1. In Vitro Pharmacology Results

Two arylpiperazine derivatives of Formula (I) were tested in the in vitro pharmacological assays to evaluate their activities for dopamine—D₁, D_{2L}, D₂ₛ, D₃, D_{4.4}; serotonin-5-HT_{1A}, 5-HT_{2A}, 5-HT_{2B}, 5-HT₆, 5-HT₇; serotonin transporter (SERT); and nicotinic acetylcholine alpha4beta2(nACh-α₄β₂)serotonin. The radioligand binding assays were carried out at six to 10 different concentrations and the test concentrations were 0.1 nM, 0.3 nM, 1 nM, 10 nm, 30 nM, 100 nM, 300 nM, 1000 nM, 10000 nM. The in vitro assay protocols and literature references are described herein.

### Dopamine, D₁ Radioligand Binding Assay

### Materials and Methods:

Receptor Source: Human recombinant D₁ expressed CHO cells

### Radioligand: [3H]SCH 23390, 0.3 nM

### Control Compound: SCH23390

Incubation Conditions: The reactions were carried out in 50 mM TRIS-HCl (pH 7.4) containing 120 mM NaCl, 5 mM KCl, 5 mM MgCl₂, 1 mM EDTA for 60 minutes at 22° C. The reaction was terminated by rapid vacuum filtration onto glass fiber filters. Radioactivity trapped onto the filters was determined and compared to control values in order to ascertain any interactions of test compounds with the cloned dopamine—D₁ binding site.

### Dopamine, D_{2L} Radioligand Binding Assay

### Materials and Methods:

Receptor Source: Human recombinant D_{2L} expressed HET-293 cells

### Radioligand: [3H]Methylspiperone, 0.3 nM

### Control Compound: Butaclamol

Incubation Conditions: The reactions were carried out in 50 mM TRIS-HCl (pH 7.4) containing 120 mM NaCl, 5 mM KCl, 5 mM MgCl₂, 1 mM EDTA for 60 minutes at 22° C. The reaction was terminated by rapid vacuum filtration onto glass fiber filters. Radioactivity trapped onto the filters was determined and compared to control values in order to ascertain any interactions of test compounds with the cloned dopamine—D_{2L} binding site.

### Dopamine, D_{2S} Radioligand Binding Assay

### Materials and Methods:

Receptor Source: Human recombinant D_{2S} expressed CHO or HEK cells
Radioligand: [³H]Spiperone (20-60 Ci/mmol) or [3H]-7-hydroxy DPAT, 1.0 nM

### Control Compound: Haloperidol or Chlorpromazine

Incubation Conditions: The reactions were carried out in 50 mM TRIS-HCl (pH 7.4) containing 120 mM NaCl, 5 mM KCl, 5 mM MgCl₂, 1 mM EDTA for 60 minutes at 25° C. The reaction was terminated by rapid vacuum filtration onto glass fiber filters. Radioactivity trapped onto the filters was determined and compared to control values in order to ascertain any interactions of test compounds with the cloned dopamine—D₂ short binding site (Literature Reference: Jarvis, K. R. et al. Journal of Receptor Research 1993, 13 (1-4), 573-590; Gundlach, A. L. et al. Life Sciences 1984, 35, 1981-1988.)

### Dopamine, D_{4.4} Radioligand Binding Assay

### Materials and Methods:

Receptor Source: Human recombinant D_{2S} expressed CHO cells

### Radioligand: [³H]Spiperone, 0.3 nM, 1.0 nM

### Control Compound: (+)Butaclamol

Incubation Conditions: The reactions were carried out in 50 mM TRIS-HCl (pH 7.4) containing 120 mM NaCl, 5 mM KCl, 5 mM MgCl₂, 1 mM EDTA for 60 minutes at 25° C. The reaction was terminated by rapid vacuum filtration onto glass fiber filters. Radioactivity trapped onto the filters was determined and compared to control values in order to ascertain any interactions of test compounds with the cloned dopamine—D_{4.4} binding site

### Dopamine, D₅ Radioligand Binding Assay

### Materials and Methods:

Receptor Source: Human recombinant D_{2S} expressed GH4 cells

### Radioligand: [3H]SCH 23390, 0.3 nM

### Control Compound: SCH 23390

Incubation Conditions: The reactions were carried out in 50 mM TRIS-HCl (pH 7.4) containing 120 mM NaCl, 5 mM KCl, 5 mM MgCl₂, 1 mM EDTA for 60 minutes at 25° C. The reaction was terminated by rapid vacuum filtration onto glass fiber filters. Radioactivity trapped onto the filters was determined and compared to control values in order to ascertain any interactions of test compounds with the cloned dopamine—D₅ binding site

### Serotonin, 5HT_{1A} Radioligand Binding Assay

### Materials and Methods:

Receptor Source: Human recombinant 5-HT_{1A} expressed mammalian cells

### Radioligand: [³H] -8-OH-DPAT (221 Ci/mmol)

### Control Compound: 8-OH-DPAT

Incubation Conditions: The reactions were carried out in 50 mM TRIS-HCl (pH 7.4) containing 10 mM MgSO₄, 0.5 mM EDTA and 0.1% Ascorbic acid at room temperature for 1 hour. The reaction was terminated by rapid vacuum filtration onto glass fiber filters. Radioactivity trapped onto the filters was determined and compared to control values in order to ascertain any interactions of test compounds with the cloned serotonin—5HT_{1A} binding site (Literature Reference: Hoyer, D. et al. Eur. Journal Pharmacol. 1985, 118, 13-23; Schoeffter, P. and Hoyer, D. Naunyn-Schmiedeberg's Arch. Pharmac. 1989, 340, 135-138)

### Serotonin, 5HT_{2A} Radioligand Binding Assay

### Materials and Methods:

Receptor Source: Human Cortex or Human recombinant 5-HT_{2A} expressed mammalian cells

### Radioligand: [³H]-Ketanserin (60-90 Ci/mmol)

### Control Compound: Ketanserin

Incubation Conditions: The reactions were carried out in 50 mM TRIS-HCl (pH 7.6) at room temperature for 90 minutes. The reaction was terminated by rapid vacuum filtration onto glass fiber filters. Radioactivity trapped onto the filters was determined and compared to control values in order to ascertain any interactions of test compounds with the serotonin—5HT_{2A} binding site (Literature Reference: Leysen, J. E. et al. Mol. Pharmacol. 1982, 21, 301-314; Martin, G. R. and Humphrey, P. P. A. Neuropharmacol. 1994, 33 (3/4), 261-273.)

### Serotonin, 5HT_{2B} Radioligand Binding Assay

### Materials and Methods:

Receptor Source: Human recombinant 5-HT_{2B} expressed CHO-K1 cells
Radioligand: 1.20 nM [3H] Lysergic acid diethylamide (LSD)

### Control Compound: Ketanserin

Incubation Conditions: The reactions were carried out in 50 mM TRIS-HCl (pH 7.6) at room temperature for 90 minutes. The reaction was terminated by rapid vacuum filtration onto glass fiber filters. Radioactivity trapped onto the filters was determined and compared to control values in order to ascertain any interactions of test compounds with the serotonin—5HT_{2B} binding site

### Serotonin, 5HT₆ Radioligand Binding Assay

### Materials and Methods:

Receptor Source: Human recombinant 5-HT₆ expressed mammalian cells

### Radioligand: [125I] SB258585, 15 nM or [³H]LSD, 2 nM

Control Compound: Methiothepin or serotonin
Incubation Conditions: The reactions were carried out in 50 mM TRIS-HCl (pH 7.4) containing 10 mM MgSO₄, 0.5 mM EDTA and 0.1% Ascorbic acid at room temperature for 1 hour. The reaction was terminated by rapid vacuum filtration onto glass fiber filters. Radioactivity trapped onto the filters was determined and compared to control values in order to ascertain any interactions of test compounds with the cloned serotonin—5HT₆ binding site (Literature Reference: Gonzalo, R., et al., Br. J. Pharmacol., 2006 (148), 1133-1143)

### Serotonin, 5HT₇ Radioligand Binding Assay

### Materials and Methods:

Receptor Source: Human recombinant 5-HT₇ expressed CHO cells
Radioligand: [3H] Lysergic acid diethylamide (LSD), 4 nM

### Control Compound: Serotonin

Incubation Conditions: The reactions were carried out in 50 mM TRIS-HCl (pH 7.6) at room temperature for 90 minutes. The reaction was terminated by rapid vacuum filtration onto glass fiber filters. Radioactivity trapped onto the filters was determined and compared to control values in order to ascertain any interactions of test compounds with the serotonin 5HT₇ binding site

### Nicotinic Acetylcholine α₄β₂ (nACh-α₄β₂) Radioligand Binding Assay

### Materials and Methods:

Receptor Source: Human recombinant nACh-α₄β₂ expressed mammalian cells

### Radioligand: [3H] Cytisine, 3.0 nM

### Control Compound: Epibatidine

Incubation Conditions: The reactions were carried out in 120 mM NaCl, 2.5 mM KCl, 50 mM Tris, 1 mM CaCl₂, 1 mM MgCl₂ containing buffer (pH 7.4) for 60 minutes at ambient temperature (37° C.). The reaction was terminated by rapid vacuum filtration onto glass fiber filters. Radioactivity trapped onto the filters was determined and compared to control values in order to ascertain any interactions of test compounds with the cloned nicotinic acetylcholine α₄β₂ (nACh-α₄β₂) binding site

### Serotonin Transporter (SERT) Radioligand Binding Assay

### Materials and Methods:

Receptor Source: Human recombinant H1 expressed mammalian cells

### Radioligand: [3H] Citalopram, 2.0 nM

### Control Compound: Venlafaxine

Incubation Conditions: The reactions were carried out in 50 mM TRIS-HCl (pH 7.4) containing 120 mM NaCl, 5 mM KCl, 5 mM MgCl₂, 1 mM EDTA for 180 minutes at ambient temperature (37° C.). The reaction was terminated by rapid vacuum filtration onto glass fiber filters. Radioactivity trapped onto the filters was determined and compared to control values in order to ascertain any interactions of test compounds with the cloned serotonin transporter (SERT) site.

| Compound | Radioligand Binding Assay | Ki (nM) |
|---|---|---|
| A | Dopamine D2S | 0.30 |
| A | Serotonin 5-HT1A | 0.65 |
| A | Serotonin 5-HT2A | 111 |
| B | Dopamine D1 | 100 |
| B | Dopamine D2L | 0.45 |
| B | Dopamine D2S | 0.28 |
| B | Dopamine D3 | 3.7 |
| B | Dopamine D4.4 | 6.0 |
| B | Serotonin 5-HT1A | 1.5 |
| B | Serotonin 5-HT2A | 2.5 |
| B | Serotonin 5-HT2B | 0.19 |
| B | Serotonin 5-HT6 | 51 |
| B | Serotonin 5-HT7 | 2.7 |
| B | Serotonin Transporter (SERT) | 107.1 |
| B | Nicotinic Acetylcholine α4β2 | 36.3 |

### Example 2. Treatment of Psychosis Associated with Parkinson's Disease

Objective: This study examines whether the compound of this invention is effective in treating psychosis associated with Parkinson's disease
Test Compound: Compound B, 6-(4-(4-(2,3-dichlorophenyl)piperazin-1-yl)butoxy)-2H-benzo[b][1,4]oxazin-3(4H)-one hydrochloride, is formulated in the form of liquid, tablet, or capsule.
Placebo contains the same vehicle without the active compound.

### Patent Inclusion Criteria

Subjects with a clinical diagnosis of idiopathic Parkinson's Disease, defined as the presence of at least three of the following cardinal features, in the absence of alternative explanations or atypical features:
Rest tremor
Rigidity
Bradykinesia and/or akinesia
Postural and gait abnormalities
Subjects with psychosis:
Presence of visual and/or auditory hallucinations, with or without delusions, occurring during the four weeks prior to the screening visit.
Symptoms severe enough to clinically warrant treatment with an antipsychotic agent.
A Hallucinations or Delusions total item score (frequency x severity) of >4 on the
Neuropsychiatric Inventory (NPI).
   Subjects currently being treated with an antipsychotic agent who have not had visual and/or auditory hallucinations, with or without delusions, during the four weeks prior to screening, and/or have a Hallucinations or Delusions total item score <4 on the NPI at the screening visit may be washed out (for 7 days or 5 half-lives, whichever is longer) and return for a repeat screening visit. The NPI Hallucinations or Delusions total item score must be ≧4 at the repeat visit to be considered for study entry.
Subject is on a stable dose of anti-Parkinsonian medication(s) for at least 7 days or 5 half-lives, whichever is longer, prior to the screening visit and is expected to remain on a stable dose for the duration of the study.
Subject is willing and able to comply with all study procedures.

### Patent Exclusion Criteria

Subject has any systemic factor contributing to the psychosis such as urinary infection, liver disease, renal failure, anemia, infection or cancer.
Subject has a history of significant psychotic disorders prior to the diagnosis of Parkinson's Disease, including but not limited to schizophrenia or bipolar disorder.
Subject has Dementia with Lewy-bodies (DLB).
Subject has dementia or a major depressive disorder precluding accurate assessment on rating scales.
Subject has an acute depressive episode at the time of the screening visit.
A score on the Mini-Mental State Examination (MMSE) of <21.
Subject has had a dose adjustment of their antidepressant medication within 30 days prior to the screening visit, or dose adjustments are planned during the duration of the trial.
Subject has had dose adjustments of an anxiolytic, cognitive enhancer, or other psychotropic medication (excluding antipsychotics) within 30 days prior to screening or dose adjustments are planned during the duration of the trial.
Subject has received depot antipsychotic agents within the past 3 months.
Subject has previously failed treatment with clozaril for psychosis in Parkinson's disease.
   Subjects who discontinued clozaril due to intolerability may be enrolled.
Subject has used any investigational product within 30 days or 5 half-lives, whichever is longer, prior to screening.
Subject cannot tolerate a wash-out of antipsychotic medication prior to randomization.
Subject has a history of a serious respiratory, gastrointestinal, renal, hematologic or other medical disorder.
Subject has a history of a serious cardiovascular condition (including, but not limited to, Class IV angina or Class IV heart failure) and/or a history of risk factors for Torsade de pointes (Tdp) (including but not limited to current treatment for hypokalemia or family history of long QT syndrome).
Subject had myocardial infarction within 6 months prior to screening.
Subject has a screening ECG with corrected QT interval by Bazett's correction formula (QTcB) of greater than 450 msec, if female, or 430 msec, if male.
Subject requires treatment with an α-agonist agent.
Subject has uncontrolled seizures, uncontrolled angina, or uncontrolled symptomatic orthostatic hypotension (or orthostatic hypotension leading to a history of falls 3 months prior to screening), or other medical disorders which would make the subject a poor candidate for a clinical trial.
Subject has a history of severe adverse reactions to antipsychotic medications and/or quinine.
Subject has clinically significant abnormal laboratory values, ECG, or findings on physical exam.
Subject has a recent history or current evidence of substance dependence or abuse.
Subject is unable to ingest liquid medication.
Subject is currently being treated with Deep Brain Stimulation (DBS).

### Randomization Criteria

Subject has a Hallucinations or Delusions total item score (frequency x severity) of >4 on the NPI.
Female subjects of childbearing potential must have a negative serum pregnancy test.
Subject has remained on a stable dose of anti-Parkinsonian medications.
Subject has not had a dose adjustment in their antidepressant medication since the screening visit.
Subjects have been washed out of previous antipsychotic agents for 5 half-lives or 7 days, whichever is longer, after the last dose of medication.
Subject has not had dose adjustments in an anxiolytic, cognitive enhancer or other psychotropic medication (excluding antipsychotics) since the Screening Visit.

### Methodology

This is placebo-controlled, double-blind treatment clinical activity study.

Treatment Duration: 1 week pretrial washout, 6 weeks of drug treatment, and 1 week post trial re-stabilization.

A total of 20-120 patients are enrolled; about ¾ of the patients are treated with Compound B, and ¼ of the patients are treated with placebo. The test compounds and the placebo are delivered either by oral administration or by transdermal patch for 8 weeks.

For oral administration, patients take 0.5-100 mg of test compound or placebo once a day.

For transdermal administration, doses that achieve similar blood concentration as that of effective oral doses are given to patients. The patches are replaced every week or every two weeks.

Patients are monitored by the treating psychiatrist. Study assessments are administered at designated time points.

### Criteria for Evaluation

### Primary Outcome Measures:

Change From Baseline in Neuropsychiatric Inventory (NPI) Psychosis Subscale Score at Week 6. The NPI is a questionnaire that quantifies behavioral changes in dementia. For each of 12 behavioral domains there are 4 scores: Frequency (scale: 1=occasionally to 4=very frequently), Severity (scale: 1=Mild to 3=Severe), Total (frequency×severity), Caregiver distress (scale: 0=not at all distressing to 5=extremely distressing). The NPI Psychosis Subscale consists of the two domains of delusions and hallucinations, calculated by adding the Individual Item Scores, to yield a possible total score of 0 to 24. Lower score=less severity. A negative change score from baseline indicates improvement.

### Secondary Outcome Measures:

Investigator/Caregiver Evaluations of Motor Function from Baseline to week 6
The change in the motor section of the Unified Parkinson's Disease Rating Scale (UPDRS III-motor exam) score. Scores on the UPDRS III-motor exam range from 0 to 108, with higher scores indicating more severe motor symptoms.
Cognition Test Battery change from baseline
   Safety measures:
   Vital signs, laboratory, ECG, physical examination

While the invention has been particularly shown and described with reference to a preferred embodiment and various alternate embodiments, it will be understood by persons skilled in the relevant art that various changes in form and details can be made therein without departing from the scope of the invention. All printed patents and publications referred to in this application are hereby incorporated herein in their entirety by this reference.

What is claimed is:

1. A method of treating psychosis associated with Parkinson's disease, the method comprising administering to a patient in need thereof an effective amount of a compound of Formula 1: 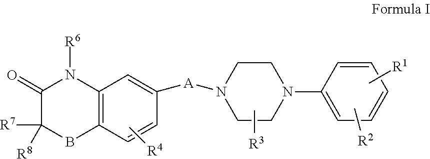
or a pharmaceutically acceptable salt, isomer, racemate, or diastereomeric mixture thereof, wherein:
A is —O—(CH₂)ₙ—, —(CH₂)ₙ—, —S—(CH₂)ₙ—, —NH—(CH₂)ₙ—, —CH₂—O—(CH₂)ₙ—, —(CH₂)ₙ———O—CH₂—CH₂—, —CH₂—S—(CH₂)ₙ—, —NH—C(O)—(CH₂)ₙ—, —CH₂—NH—C(O)—(CH₂)ₙ—, —CH₂—C(O)—NH—(CH₂)ₙ—, or —(CH₂)ₙ—C(O)—NH—CH₂—CH₂—, wherein n is an integer from 1 to 7;
B is O, S, S(O)(O), or NRS; and
R¹, R², R³, R⁴, R⁶, R⁷, and R⁸ are independently hydrogen, alkyl, substituted alkyl, aryl, substituted aryl, arylalkyl, substituted arylalkyl, cycloalkyl, substituted cycloalkyl, alkoxy, alkoxycarbonyl, alkylsulfinyl, alkylsulfonyl, alkylthio, amino, alkylamino, dialkylamino, arylalkoxy, carboxy, carbamoyl, carbamate, carbonate, cyano, halogen, or hydroxy; wherein the hydrogen of R¹, R², R³, R⁴, R⁶, R⁷and R⁸ and A are optionally substituted with ²H (deuterium).

2. The method according to claim 1, wherein A is —O—(CH₂)ₙ—.

3. The method according to claim 1, wherein A is —(CH₂)ₙ—.

4. The method according to claim 1, wherein A is —NH—C(O)—(CH₂)ₙ—, —CH₂—NH—C(O)—(CH₂)ₙ—, —CH₂—C(O)—NH—(CH₂)ₙ—, or —(CH₂)ₙ—C(O)—NH—CH₂—CH₂—.

5. The method according to claim 1, wherein B is O.

6. The method according to claim 1, wherein R³, R⁴, R⁶, R⁷, and R⁸ are hydrogen.

7. The method according to claim 6, wherein R¹ and R² are independently H, halogen, or alkoxy.

8. The method according to claim 6, wherein R¹ is H, and R² is methoxy.

9. The method according to claim 6, wherein R¹ and R² are chloro.

10. The method according to claim 1, wherein A is —O—(CH₂)ₙ—; B is O, and R³, R⁴, R⁶, R⁷, and R⁸ are independently hydrogen or alkyl.

11. The method according to claim 1, wherein the compound is 6-(4-(4-(2,3-dichlorophenyl)piperazin-1-yl)butoxy)-2H-benzo[b][1,4]oxazin-3(-4H)-one, or its hydrochloride salt thereof.

12. The method according to claim 1, wherein the compound is 6-(4-(4-(2-methoxyphenyl)piperazin-1-yl)butoxy)-2H-benzo [b][1,4] oxazin-3(-4H)-one, or its hydrochloride salt thereof.

13. The method according to claim 1, wherein the compound is administered in a pharmaceutical composition comprising a pharmaceutically acceptable carrier, excipient, or diluent.

14. The method according to claim 1, wherein the compound is orally administered.

15. The method according to claim 1, which treats dementia.

16. The method according to claim 1, which treats memory impairment and/or cognitive impairment in the patient.

17. The method according to claim 1, which treats agitation in the patient.

18. The method according to claim 1, which treats depression in the patient.

19. The method according to claim 1, which treats mood swing in the patient.
